(19) United States
(12) Patent Application Publication
(10) Pub. No.: US 2020/0197309 A1
(43) Pub. Date: Jun. 25, 2020

(54) METHODS AND COMPOSITIONS FOR THE TREATMENT OF RETINAL, NEUROLOGICAL AND OTHER RELATED DISEASES

(71) Applicant: Verdure Sciences, Noblesville, IN (US)

(72) Inventors: Lal Hingorani, Noblesville, IN (US); Sonya Cropper, Noblesville, IN (US)

(21) Appl. No.: 16/645,933

(22) PCT Filed: Sep. 11, 2017
(86) PCT No.: PCT/US2017/050941

§ 371 Date: Mar. 10, 2020

(51) Int. Cl. A61K 9/16; A61K 9/00; A61K 31/047; A61K 31/12; A61K 47/24; A61P 29/00; A61P 27/02

(52) U.S. Cl. CPC A61K 9/167; A61K 9/0053; A61K 31/047; A61P 27/02; A61K 9/1617; A61K 47/24; A61P 29/00; A61K 31/12

(57) ABSTRACT

The present invention provides compositions and formulations of biologically active compounds complexed with a solid lipid particle which is able to traverse the blood-retinal barrier and treat retinal and neurological diseases.

### CROSS-REFERENCE TO RELATED APPLICATIONS

N/A

### FIELD OF THE INVENTION

The present invention generally relates to lipophilic compounds, particularly solid lipid particles useful for treatment of retinal and neurological diseases.

### BACKGROUND OF THE INVENTION

Inflammation is a characteristic of a number of diseases and disorders, including ocular and neurological diseases and disorders. Chronic or acute eye inflammation, including retinal inflammation, may give rise to a number of diseases which can result in permanent loss of vision. As such, there has been a push to find new methods and compositions for reducing or eliminating chronic retinal inflammation in order to reduce or inhibit permanent retinal damage and/or vision impairment. Retinal degenerative diseases are the major cause of vision loss and blindness worldwide and characterized by chronic and progressive neuronal loss. Retinal degenerative diseases may be characterized by chronic inflammation. The retina, which is part of the central nervous system, is structured in nuclear layers of neurons. The retina includes, in part, the outermost layer of retinal pigment epithelium (RPE), followed by the outer nuclear layer (ONL) which contains the cell bodies and photoreceptors, the inner nuclear layer (INL) which contains the cell bodies of the bipolar, horizontal, and amacrine cells, and the ganglion cell layer (GCL) composed by the nuclei of retinal ganglion cells (RGCs). These cellular layers are interconnected through synapses occurring in the outer and inner plexiform layers. Besides neurons, other cells are present in the retina, such as glial cells (Müller cells, astrocytes, and microglia) and the cells that constitute the retinal vessels (endothelial cells and pericytes). See e.g., Maria H. Madeira, Raquel Boia, Paulo F. Santos, António F. Ambrósio, and Ana R. Santiago, “Contribution of Microglia-Mediated Neuroinflammation to Retinal Degenerative Diseases,” Mediators of Inflammation, vol. 2015, Article ID 673090, 15 pages, 2015. doi:10.1155/2015/673090\. The retina comprises a complex interplay of neuronal cells that when inflamed, break down the connectivity resulting in neuronal cell death and permanent vision loss.

As a turmeric extract, curcumin (diferulomethane) is the yellow seen in yellow curries and is used as a food additive, for example, in yellow mustard. Like the “wonder drug” aspirin, which remains one of our few successful preventive agents, curcumin has been identified as a bioactive agent in an empirically developed system of traditional Indian and Chinese medicine.

Curcumin is not only a potent natural antioxidant and anti-inflammatory agent, acting on NFKB and AP-I regulated pro-inflammatory mediators including COX-2, iNOS, il-I and TNFα, but has multiple useful activities and has demonstrated therapeutic potential in many pre-clinical culture and animal models for diseases, often related to aging. These include cancers (colon, prostate, breast, skin, leukemia, etc.) (Agarwal et al., 2003), prion disease (Caughey et al., 2003), atherosclerosis (Miguel et al., 2002; Ramaswami et al., 2004), stroke, CNS alcohol toxicity (Rajakrishnan et al., 1999), traumatic brain injury, Huntington's disease, Marie-Charcot Tooth, multiple sclerosis, and Alzheimer's disease.

Curcumin's structure, which includes both a lipophilic moiety and at least one hydroxyl group, resembles that of amyloid binding compounds. However, use of curcumin for therapeutic applications is limited due to poor intestinal absorption and an inability to target specific areas of inflammation within the patient.

Although curcumin is an effective medication in multiple animal models for human diseases when given in chow at high doses (typically 2,000-5,000 ppm in diet in cancer trials), it is so poorly bioavailable that it cannot be used for treatment outside the colon in humans. Curcumin is very hydrophobic and typically is not dissolved when delivered as a powder extract in common nutraceuticals. Most curcumin activities require 100-2,000 nanomolar (0.1-2 micromolar) levels in vitro, but current supplements result in negligible, low nanomolar blood levels (See Garcea G., Jones J. D., Singh R., Deunison A. R., Farmer P. B., Sharma R. A., Steward W. P., Gescher A. J., Berry D, P., “Detection of curcumin and its metabolites in hepatic tissue and portal blood of patients following oral administration,” Br J Cancer, 2004 Mar. 8; 90 (5); 1011-5, and Sharma et al, “Phase I Clinical Trial of Oral Curcumin”, Clinical Cancer Research, 2004, Oc. 15 (10),; 6847-6854) (hereinafter “Sharma et al., 2004”). Sharma's group has attempted repeatedly and has been unable to achieve significant blood levels beyond the low nanomolar range (see Sharma et al., 2004) Sharma and others conclude that delivery of effective concentrations of oral curcumin to systemic tissues (outside the GI tract) is not feasible. Most literature supports this view, leading the National Cancer Institute (NCI) to focus on colon cancer.

Curcumin is not soluble at acidic pH and breaks down when solubilized and diluted into water at neutral or alkaline pH (e.g., in the GI tract, after the small intestine), due to keto-enol transformations in the β-diketone bridge. In addition, curcumin is susceptible to rapid glucuronidation/sulfation. Suppliers have attempted to develop a more bioavailable form by adding piperine to inhibit glucuronidation. Such an approach is flawed, however, because most glucuronidation takes place in the upper GI tract, where the pH is acidic, and curcumin is not completely dissolved until at least a pH of 8.5 or higher. In addition, inhibiting glucuronidation can cause serious health risks. Glucuronidation is protective against many toxins and involved in the metabolism of commonly used drugs. Most elderly patients are on multiple drugs, at levels likely to be unfavorably, and unsafely, altered by the inhibition of glucuronidation.

Curcuminoids are but one example of lipophilic compounds which possess both a lipophilic moiety and at least one hydroxyl group. These compounds in general have difficulty with bioavailability as well as stability in an oral dosage form. Oral bioavailability requires at a minimum stability, solubility and permeability of the active compound in the gut; however lipophilic compounds are generally not water soluble, and lipophilic compounds with hydroxyl groups may possess hydrolytic instability. Such solubility and instability issues are a substantial problem, both for bioavailability in the subject and for stability of the dosage form both in the gut and on the shelf.

There is a need for methods and formulations that can reduce inflammation within the eye, specifically formulations and compounds which are able to traverse the blood-ocular barrier and possess anti-inflammatory properties.

### DESCRIPTION OF THE DRAWINGS

FIG. 1 is a scanning electron micrograph taken at 10 Kv and 170× magnification of a 30 mesh carrier granule having a plurality of spherical solid lipid particles (SLPs) embedded in or adhering to the surface of the granule.

FIG. 2 is a graph demonstrating solid lipid particle carrier granule complex (SLCP) containing curcumin dose-dependent inhibition of prostaglandin production. A comparison of (a) free curcumin vs. (b) SLCP curcumin is shown.

FIG. 3 is a graph demonstrating plasma concentrations of free curcumin (standard and SLCP) and glucuronated curcumin (standard and SLCP)(mean values±s.d.; n=5).

FIG. 4 is a graph showing lymphatic fluid concentrations of curcumin (standard) and curcumin (SLCP) (mean values±s.d; n=5).

FIG. 5 is a graph showing plasma lutein concentrations before (pre) and after 10 days supplementation with 10 mg/day of lutein (post). Lutein 1=lutein ester. Lutein 2=solid-lipid particle (SLCP) complex lutein. *Significantly different from pre value, p<0.001, paired t-test.

FIG. 6 is a graph showing the change in plasma lutein concentrations after 10 days of supplementation with 10 mg/day of lutein. Lutein 1=lutein ester. Lutein 2=solid-lipid particle (SLCP) complexed lutein. *Significantly different from pre value, p<0.002, unpaired t-test.

FIG. 7 is a graph showing plasma lutein concentrations 7 days post-supplementation (10 mg/day of lutein for 10 days). Lutein 1=lutein ester. Lutein 2=solid-lipid particle (SLCP) complex lutein. * Significantly different from pre value, p<0.001, unpaired t-test.

FIG. 8 is representative SEM images of empty solid lipid particles (SLPs) and carrier granules.

FIG. 9 is representative SEM images of curcumin SLP (for the purposes of this disclosure is also known as Longvida®) complexed with carrier granules.

FIG. 10 is representative SEM images of curcumin SLP (Longvida®) in powder form.

FIG. 11 is representative SEM images of curcumin SLP Longvida® solution dispersible (SD).

FIG. 12A shows stereological quantification of 18 kDA translocator protein (TSPO) in the cerebellum of 24 month-old GFAP-IL6 and WT mice. In the cerebellum, there was significantly more TSPO positive microglia in the GFAP-IL6 mice (4.75 times more than WT).

FIG. 12B shows stereological quantification of TSPO in the hippocampus of 24-month old GFAP-IL6 and WT mice. In the hippocampus there was significantly more TSPO positive microglia in the GFAP-IL6 mice (2.28 times greater than WT).

FIG. 12C shows a representative immunohistochemical stain of thyroid peroxidase (TPO) positive microglia in the cerebellum of a 3 month old WT mouse.

FIG. 12D shows a representative immunohistochemistry stain of TPO positive microglia in the cerebellum of 3 month old GPAP-IL6 mouse.

FIG. 13 shows evaluation of brain inflammation using autoradiography with the TSPO ligand [125I]-Clinde (TPSO ligand). Autoradiography with [125I]-Clinde in WT and GFAp-IL6 mice. Note that binding appears to be increased in the GFAP-IL6 cerebellum compared to WT mice (n=1 each).

FIG. 14A shows representative fluorescent images of lba-1 positive microglia (marker of activated microglia) for WT or GFAP-IL6 mice at 3 or 24 months.

FIG. 14B depicts the quantitated number of activated microglia in the WT versus GFAP-IL6 mice.

FIG. 15 is the Fourier transform infrared spectroscopy (FTIR) plot for empty SLPs.

FIG. 16 is the FTIR plot overlay of different SLP formulations.

FIG. 17 is the FTIR plot for overall spectra of curcumin.

FIG. 18 is the FTIR plot for lutein.

FIG. 19 is the FTIR plot overlaying Longvida® (curcumin) and lutein.

FIG. 20 is the FTIR plot for Longvida® SD showing all ingredients.

FIG. 21 is the FTIR plot for Longvida® showing all ingredients.

### SUMMARY OF THE INVENTION

The present invention provides formulations and compositions for use in the treatment of retinal and neurological disorders. The formulations have improved stability, solubility and permeability in the gut after oral consumption, resulting in parent (native) compound levels that are therapeutic (as opposed to inactive metabolites such as glucuronides) in specific tissues. The formulations and compositions are used to treat illnesses relating to inflammation of the eye, including retinal inflammation or oxidation where a therapeutic blood and tissue level of the active is required for treating the illness. Further the formulations of certain embodiments of the present disclosure are able to cross the blood-ocular barrier, including the blood-retinal barrier, allowing for methods of treating retinal inflammation with therapeutically effective tissue levels.

An embodiment of the present disclosure comprises a method of treating, reducing or inhibiting retinal inflammation in a subject, the method comprising: administering an effective amount of an enhanced oral bioavailable formulation of curcumin, lutein or a combination thereof, the enhanced oral bioavailable formulation comprising: (a) curcumin, lutein or a combination thereof complexed with a solid lipid particle; (b) a carrier granule comprising an agglomeration of the solid lipid particle embedded in, adhered to or both embedded in and adhered to, the carrier granule, wherein the solid lipid particle carrier granule complex is formulated for oral dosing.

An embodiment of the present disclosure comprises a method of treating a retinal disease in a subject, the method comprising: administering an effective amount of an enhanced oral bioavailable formulation of curcumin, lutein or a combination thereof, the enhanced oral bioavailable formulation comprising: (a) curcumin, lutein or a combination thereof complexed with a solid lipid particle: (b) a carrier granule comprising an agglomeration of the solid lipid particle embedded in, adhered to or both embedded in and adhered to, the carrier granule, wherein the solid lipid particle carrier granule complex is formulated for oral dosing. In an embodiment the retinal disease may be selected from the group consisting of glaucoma, diabetic retinopathy, and retinal vein occlusion. In an embodiment the retinal disease may be associated with retinal inflammation.

In an embodiment the carrier granule has a particle size from about 150 to about 840 microns. In an embodiment the solid lipid particle has a particle size from about 5 to about 20 microns. In an embodiment the solid lipid particle comprises one or more long-chain lipid. In an embodiment the one or more long chain lipid may be selected from the group consisting of soy lecithin, phosphatidyl choline, stearic acid, and ascorbyl palmitate. In an embodiment the solid lipid particle may further comprise dextrin, silicone dioxide, or both. In an embodiment the ratio steric acid: phosphatidyl choline is in a range of about 1.25:1 to about 3.5:1, or greater than about 3:5:1.

In an embodiment the solid lipid particle comprises a core comprising the active ingredient, phosphatidylcholine and ascorbyl palmitate and a coating of stearic acid. In an embodiment the solid lipid particle is embedded in the carrier granule. In an embodiment the solid lipid particle is adhered to the surface of the carrier granule. In an embodiment the solid lipid particle is encased within the carrier granule. In an embodiment the carrier granule may be formed in all or in part from fractured solid lipid microparticles. In an embodiment at least one of the carrier granule, solid lipid particle, and carrier granule solid lipid particle complex pass through the blood-ocular barrier.

In an embodiment the formulation comprises: about 15 to about 40% of curcumin, lutein, or a combination thereof; about 7 to about 25% soya lecithin; about 7 to about 30% maltodextrin; about 1 to about 3% ascorbyl palmitate; and about 0.3 to about 2% silicone dioxide. In an embodiment the soya lecithin may be phosphatidyl choline. In an embodiment the formulation comprises: about 10 to about 30% of curcumin, lutein or a combination thereof; about 10 to about 20% of phosphatidylcholine; about 25 to about 35% stearic acid; about 25 to about 40% dextrin; about 1 to about 4% ascorbyl palmitate; and about 0.1 to about 3% silicon dioxide.

### DETAILED DESCRIPTION OF THE INVENTION

Embodiments of the present disclosure provide methods of treating diseases of the eye or brain, preferably diseases associated with retinal inflammation. In some embodiments, curcumin and/or lutein are provided in a delivery system which is comprised of an agglomeration of solid lipid particles (SLPs) for oral dosing, comprising a specific ratio of long-chain lipids. The lipids possess a balance of positively and negatively charged groups ideal for stabilizing lipophilic compounds containing at least one hydroxyl group. Such lipids useful for the SLP include, but are not limited to, high-purity phosphatidylcholine (i.e. charged phospholipid), stearic acid (i.e. electrostatically balancing fatty acid), and ascorbyl palmitate (amphiphilic antioxidant), in a specific ratio or approximate range of ratios, as discussed further below. Certain formulations of the SLP that can be used in the some embodiments of the current invention are disclosed in PCT Application No. PCT/US2015/060143 filed Nov. 11, 2105, which is incorporated by reference in its entirety.

In one embodiment, an oral dosage formulation may comprise an active ingredient of curcumin, lutein or a combination of both, phosphatidylcholine, stearic acid, and ascorbyl palmitate. Dextrin and silicon dioxide may also be used. In accordance with a further embodiment, the oral dosage may include the individual components in relative parts as follows:

Active compound: about 10 to about 30% (curcumin, lutein or a combination of both)

Phosphatidylcholine: about 10 to about 20%

Stearic acid: about 25 to about 35%

Dextrin: about 25 to about 40%

Ascorbyl palmitate: about 1 to about 4%

Silicon dioxide: about 0.1 to about 3%

Suitably, the oral dosage formulation is prepared using a ratio of stearic acid to phosphatidylcholine (PC) that exceeds 1. In certain embodiments, the ratio of stearic acid: PC is in a range of about 1.25:1 to about 3.5:1 or greater than about 3.5:1. In another embodiment, the oral dosage may include the individual components in relative parts as follows:

Active compound: about 15 to about 40% (curcumin, lutein or a combination of both)

Soya lecithin: about 7 to about 25%

Maltodextrin: about 7 to about 30%

Ascorbyl palmitate: about 1 to about 3%

Silicon dioxide: about 0.3 to about 2%

In some embodiments, the soya lecithin is phosphatidylcholine.

The oral delivery system is suitably in the form of an agglomeration or plurality of SLPs containing curcumin, lutein or a combination thereof embedded in or adhering to a carrier granule.

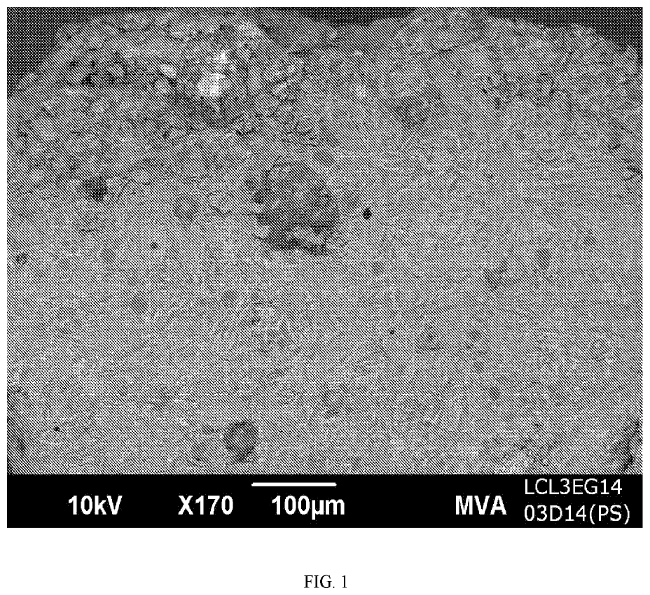

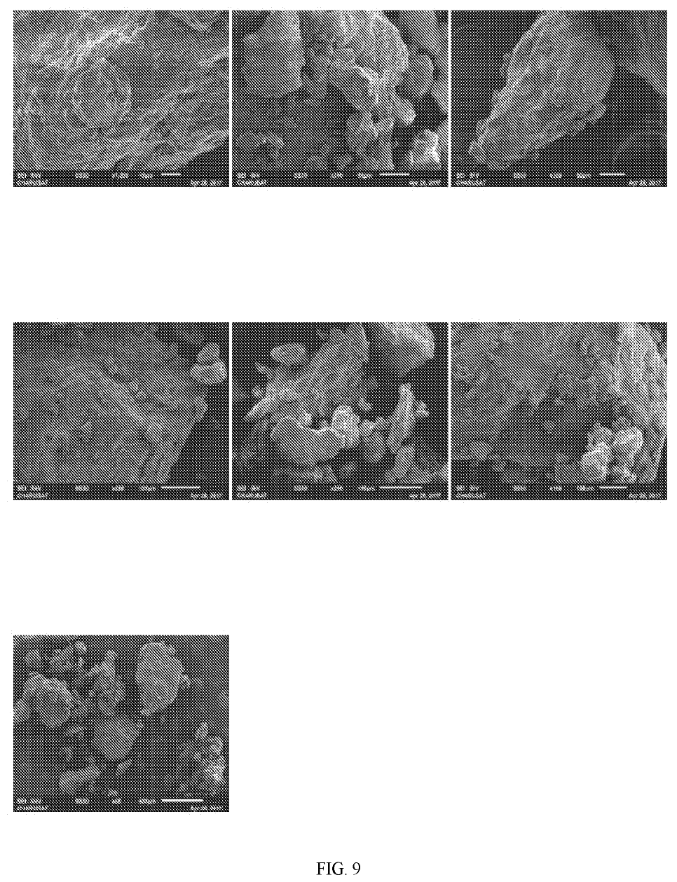

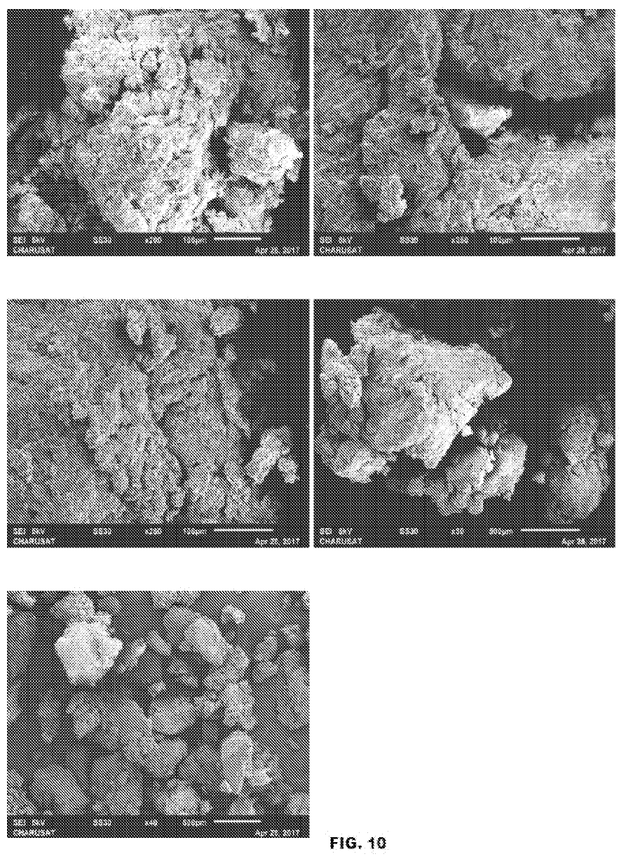

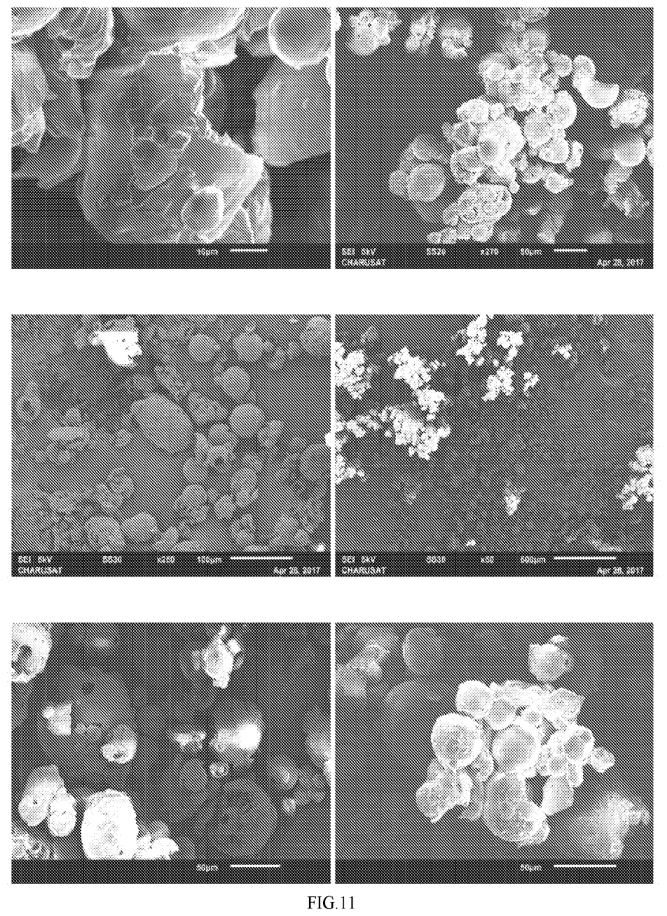

For example, as shown in FIG. 1, a plurality of SLPs having a core of an active ingredient, including curcumin and/or lutein, or a combination of both, and a coating of stearic acid are embedded in or adhered to a carrier granule that acts as a carrier for the microparticles. In accordance with certain embodiments, the carrier granules have a particle size in the range of about 20 to about 90 mesh (i.e., between about 150 microns and about 840 microns). The SLP has a particle size in the range of about 5 to about 20 microns. The SLP and/or SLPs can be found in granules or powder form. In accordance with certain other embodiments, the carrier granules may further include at least one SLP fully encased within the carrier granule matrix. The carrier granule may be formed from fractured SLPs produced during the manufacturing process. FIG. 9 shows representative SEM images of the SLP carrier granule complex (SLCP) containing curcumin, which are from about 10 to about 500 microns in size. Suitable sizes of SLCPs containing curcumin for use in embodiments of the present disclosure include, but are not limited to, SLCPs from about 10 to about 500 microns, alternatively from about 10 to about 400 microns, alternatively from about 10 to about 300 microns, alternatively about 10 to about 250 microns, alternatively from about 10 to about 100 microns, alternatively from about 10 to about 50 microns, alternatively from about 50 to about 500 microns, alternatively from about 100 to about 500 microns, alternatively from about 250 to about 500 microns in size, and include ranges and amounts in-between (e.g. about 15, about 20, about 25, about 50, about 75, about 100, about 125, about 150, about 200, about 250, about 275, about 300, about 325, about 350, about 400, about 450, about 475, about 500 microns). In some embodiments, the curcumin SLPs are in a powder form, wherein the powder is comprised of an agglomeration of SLPs encapsulating curcumin, wherein the size of the powder particles are from about 100 to about 500 microns in size. FIG. 10 depicts representative SEM images of curcumin SLP powder of an embodiment of the present invention. For example, FIG. 11 depicts representative SEM images of LONGVIDA® SD (solution dispersible) product. Suitable sizes of powder SLP curcumin for use in embodiments of the present invention include, but are not limited to, particles from about 100 to about 500 microns, alternatively about 100 to about 250 microns, alternatively from about 100 to about 200 microns, alternatively from about 150 to about 500 microns, alternatively from about 200 to about 500 microns, alternatively from about 250 to about 500 microns, alternatively from about 300 to about 500 microns in size, and include ranges and amounts in-between (e.g. about 100, about 125, about 150, about 175, about 200, about 210, about 225, about 250, about 300, about 350, about 375, about 400, about 425, about 450, about 470, about 475, about 500 microns, etc.).

Without being bound thereby, it is believed that by providing an agglomeration or plurality of SLPs embedded in, adhered or embedded in and adhered to a carrier granule (SLCP), the delivery system acts to protect the hydroxyl group of the active ingredient from hydrolysis in the gut thus improving the stability of the active ingredient. It is also believed that the oral delivery system encourages direct lymphatic absorption of the lipophilic active ingredient through the chylomicron thereby avoiding first pass metabolism in the liver (i.e., glucuronidation) and increasing bioavailability of the active ingredient.

The formulations of embodiments of the present disclosure improve the stability, solubility, and permeability of cucumin and/or lutein in the gut after oral dosing, resulting in therapeutic levels in the targeted tissues (as opposed to inactive metabolites such as glucuronides) that can be used to treat retinal inflammation or other eye disorders and/or diseases resulting from retinal inflammation.

Curcumin and/or lutein or combinations thereofin the formulations described herein may be used to treat illnesses relating to inflammation or oxidation within the eye (e.g. neuronal or retinal cells) where a therapeutic blood and tissue level of the active within the eye is required for treating the illness.

Specifically, the formulations may be used to transport drugs across the blood-ocular barrier, including the blood-retinal barrier to increase levels of the biologically active agent in the eye. The blood ocular barrier is a barrier created by endothelium of the capillaries of the retina and iris, ciliary epithelium and retinal pigment epithelium creating a physical barrier between the local blood vessels and most parts of the eye itself. The blood-ocular barrier includes the blood-aqueous barrier (the ciliary epithelium and capillaries of the iris) and the blood-retinal barrier (BRB), consisting of retinal vascular endothelium and the retinal pigment epithelium to produce a physiological barrier comprising a single layer of non-fenestrated endothelial cells which have tight junctions between retinal epithelial cells preventing passage of large molecules from choriocapillaries into the retina. As demonstrated in the examples below, the formulations according to embodiments of the present invention are able to traverse the blood-ocular barrier, including blood-retinal barrier allowing for specific delivery of the biologically active component to the eye. Suitable curcumin, lutein or combinations of curcumin and lutein can be delivered by SLP to the eye of a patient in need of such treatment, wherein there is local accumulation of the curcumin, lutein or combination thereof to achieve a therapeutically effective amount that reduces the local inflammation, for example retinal inflammation within the eye.

Suitably, the formulations of SLPs containing curcumin, lutein or combinations of curcumin and lutein can be used to treat a subject having a retinal degenerative disease. Suitable retinal degenerative diseases that can be treated by the methods and compositions include, but are not limited to, glaucoma, age-related macular degeneration (AMD), diabetic retinopathy, and retinal vein occlusion, among others. These retinal diseases are characterized by chronic neuroinflammation. Overactivation of microglial cells results in excessive production of inflammatory mediators which leads to the initiation and perpetuation of the inflammatory response. The accumulation of inflammatory mediators is harmful to neurons, further contributing to retinal neurodegeneration. If untreated, this inflammation results in neuronal cell death that can result in visual impairment and blindness. In certain embodiments the compositions and formulations of SLP and curcumin, lutein, or combinations thereof of the present invention can be used in methods that reduce, inhibit or prevent the overactivation of microglia, resulting in a reduction or inhibition of the production of inflammatory markers. In turn, this leads to a reduction or inhibition of neuroinflammation within the eye.

The oral dosage formulation demonstrates increased solubility and stability and the ability to traverse the blood-ocular barrier. This allows for therapeutically effective amounts of the biologically active ingredient to accumulate in the eye where the treatment is needed.

Suitably, the compositions and formulations to treat retinal inflammation and neuroinflammation within the eye include, but are not limited to at least one SLP comprising curcumin embedded on, adhered to or both embedded in and adhered to, the surface of a carrier granule forming a (SLCP), at least one SLP comprising lutein embedded on or adhered to the surface of a carrier granule (SLCP), and/or at least one SLP comprising a combination of curcumin and lutein embedded on or adhered to or both in and to the surface of a carrier granule (SLCP), as described in more detail herein.

The term “treat”, “treating”, and “treatment” as used herein includes, but is not limited to, reducing or inhibiting an inflammatory response, including neuroinflammation and retinal inflammation, reducing, inhibiting or preventing one or more symptoms of a retinal or neurological disease, reducing or inhibiting activation of microglia within the eye and/or reducing or inhibiting the release of inflammatory markers by microglia in the eye. In some embodiments, the term “treating” includes reducing or inhibiting the loss of vision or loss of neuronal or retinal cells caused by an inflammatory retinal disease as described herein. In some embodiments, the reducing or inhibiting loss of vision refers to, for example, reducing or inhibiting the loss of peripheral vision, reduction or inhibition of floaters, shadows or missing areas of vision, night-blindness, loss of center vision and/or other vision impairments or loss.

The term “effective amount” or “therapeutically effective amount” is an amount that provides an anti-inflammatory benefit to the eye or retina. Suitably, an effective amount can be measured by a reduction in activated microglia in the eye, reduction in inflammatory markers within the eye, and/or reduction in one or more symptom of retinal diseases, including, for example, a reduction or inhibition of neuronal or retinal cell death.

The term “subject” and “patient” are used herein interchangeably to refer to a mammal, preferably a human.

The formulations and compositions described herein for curcumin and lutein and combinations thereof can be used in the methods for treating retinal and neurological diseases.

Table 1 demonstrates the solubility data on curcumin SLPs embedded in, or adhered to or both in and to, the surface of a carrier granule (SLCP)**:

TABLE 1

| | Solubility | Solubility | |
|---|---|---|---|
| Test | in water | in water | Fold |
| formulation | % | (μg/mL) | improvement |
| Curcumin | 0.00006 | 0.6 | 1 |
| (unformulated)* | | | |
| SLCP-1 | 14 | 140,000 | 233000 |
| SLCP-2 | 76 | 760,000 | 1270000 |

*Biji T. Kurien, Anil Singh, Hiroyuki Matsumoto, and R. Hal Scofield, Improving the Solubility and Pharmacological Efficacy of Curcumin by Heat Treatment ASSAY and Drug Development Technologies. August 2007, Vol. 5, No. 4: 567-576 source: http://online.liebertpub.com/doi/abs/10.1089/adt.2007.064
** 5% w/v in DI water at 37° C.
Note:
SLCP-1 is ~30 mesh (about 595 microns) while SLCP-2 is ~80 mesh (about 175 microns) powder (Mesh = number of squares per linear inch).
In one embodiment, the oral delivery system including an active compound may be prepared using the following steps:
(1) Complexing the active ingredient in solution with purified PC (80-90% phosphatidylcholine and ascorbyl palmitate;
(2) Homogenizing the complex from Step (1) at high speed with additional ascorbyl palmitate, dextrin, and, optionally, silicon dioxide;
(3) Filtering and spray drying the homogenized complex;
(4) Mixing the spray-dried powdered complex with heated (melted) stearic acid at high speed; and
(5) Cooling and then milling the material from Step (4) to a powder including granules having agglomerated and/or a plurality of SLPs embedded in or adhered to or both in and to the surface of the granules.

Any solvent suitable for dissolving the active ingredient(s), the phosphatidyl choline, and the ascorbyl palmitate may be used in Step (1). In accordance with embodiment, the solvent used in Step (1) may include ethyl acetate.

The oral delivery system disclosed herein addresses several issues associated with the oral therapeutic use of active ingredients having both a lipophilic moiety and at least one hydroxyl group.

PROBLEM: One of the major challenges with dissolved compounds is their permeability through cell membranes. This may be due to inadequate charge on the active compound. Embodiments of the present invention solve such problems.

PROOF: Presence of phosphatidylcholine, stearic acid, ascorbyl palmitate, all long-chain lipids.

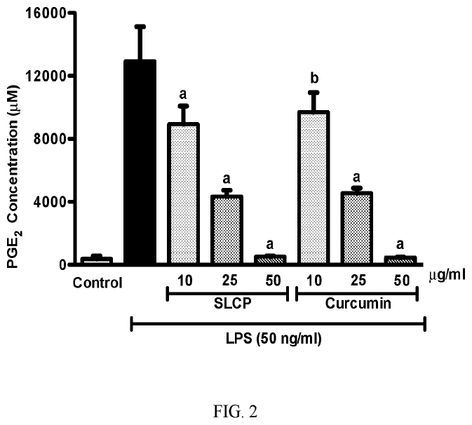

SLCPs are permeable into cultured cells where they dose-dependently inhibit prostaglandin production after stimulation with lipopolysaccharide (LPS) as shown in FIG. 2. (Data obtained October 2013, unpublished).

### Example 1: Curcumin Formulations

PROBLEM: Compounds that initially achieve solubility, stability and permeability upon preparation and dosing are rapidly metabolized by the liver into inactive conjugated metabolites. Curcumin is insoluble in water at neutral and acidic pH, and rapidly hydrolyzes or breaks down in the alkaline conditions of the small intestine. Once consumed, curcumin fails to fulfill the three major requirements for bioavailability: solubility, permeability, and stability. Approaches to address this issue have not resulted in finding detectable levels of curcumin in the body. Products that mix curcumin in oils, process it into micronized or nanoparticles, or add piperine have all failed to result in blood levels of curcumin.

Embodiments of the present disclosure allow for increased uptake of active compound onto the chylomicron and into the lymphatic system, allowing rapid exposure to cells. When curcumin is combined with these SLCPs in a specific formulation, it becomes dissolvable in the small intestine, protected from the alkaline environment and adsorbed onto the chylomicron.

Data showing preferential uptake of curcumin into lymph fluid with this formulation, after single oral dosing by rodents, is described below (unpublished).

Following previously known methods, a total of ten Sprague-Dawley rats received a single oral administration of 75 mg curcumin/kg body weight as either a standard aqueous suspension (Curcumin from Sigma # C7727) or solid lipid curcumin microparticles plus carrier granule (SLCP) prepared as described above. Prior to treatment, the rats were anaesthetized followed by a surgical intervention to cannulate the mesenteric lymph duct (for lymph collection) and the carotid artery (for blood collection). The cannulas were externalized and the rats were allowed to recover for 24 hours. Thereafter, either the curcumin suspension or the SLCP dosage form was administered via gavage. Blood (0.25 ml) was sampled prior to and 0.5, 1.0, 2.0, 3.0 and 5.0 hours after administration into vials coated with EDTA. Blood samples were centrifuged to obtain plasma and stored at <−30° C. until analysis. Mesenteric lymph (0.25 ml) was collected prior to and 1 and 5 hours after administration into vials coated with EDTA and stored at <−30° C. until analysis.

Analysis of curcumin and glucuronated curcumin was performed by HPLC with UV-detection coupled to MS according to published methods. The analytical method was validated including stability of curcumin and curcumin glucuronide in blood/plasma and lymphatic fluid. The Limit of Determination was set to 2.5 ng/ml biological fluid.

Results and Discussion:

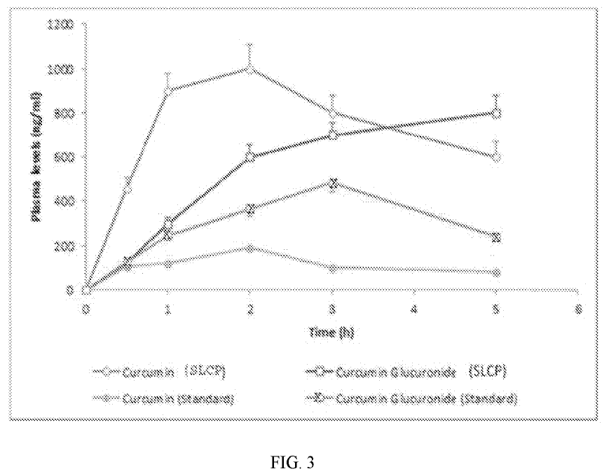

As seen in FIG. 3 and Table 2, the plasma concentration of curcumin is substantially higher after administration of the SLCP dosage form when compared to the standard curcumin suspension. In addition, the ratio of free to glucuronated curcumin indicates that after administration of the SLCP dosage form a substantial higher fraction of free curcumin reaches the blood stream when compared to the standard curcumin suspension. The pharmacokinetic test results suggest a 5-fold higher relative bioavailability of curcumin after administration of the SLCP dosage form when compared to the standard curcumin suspension.

TABLE 2

| | |
|---|---|
| Curcumin | Curcumin Glucuronide |

| Parameter^{†} | SLCP | Standard | SLCP | Standard |
|---|---|---|---|---|
| Cₘₐₓ (ng/ml) | 1000 | 190 | 800 | 483 |
| AUC₀₋ₜ (ng × | 3704 | 562 | 2736 | 1579 |
| ml/h) | | | | |

†Cₘₐₓ: Maximum concentration observed;
AUC₀₋ₜ: Area Under the plasma concentration/time Curve from time 0 to the last plasma concentration determined

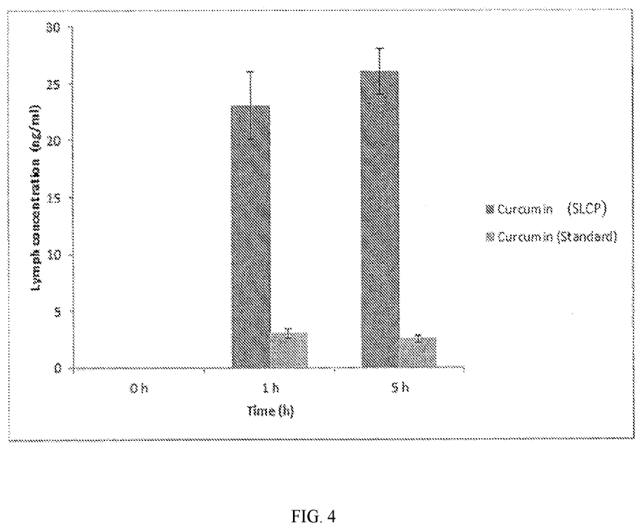

In FIG. 4 the lymphatic concentrations of curcumin are shown. About 10-fold higher concentrations of curcumin were observed after administration of SLCP complexed curcumin when compared to the standard curcumin suspension.

The high amount of curcumin found in the lymphatic fluid after treatment with SLCP complexed curcumin demonstrates its increased bioavailability when compared to the standard curcumin suspension.

It is well known that curcumin undergoes extensive first-pass metabolism yielding high amounts of curcumin-glucuronide in the blood after oral administration. The lymphatic transport of curcumin after oral administration in the SLCP dosage form circumvents the liver and the first-pass metabolism. An increased level of curcumin transport through the lymphatic system prompts the delivery of higher amounts of curcumin into the blood stream.

### Example 2: Lutein

PROBLEM: Lutein (lipophilic compound with hydroxyl group) is insoluble in water, and the portion of lutein which is soluble rapidly degrades upon exposure to water.

SOLUTION: The technology disclosed herein stabilizes actives in this class.

Human Lutein Bioavailability data (unpublished) demonstrates an increase in the bioavailability of lutein formulated as in the oral delivery system disclosed above (Lutein SLP) (Post) versus conventional lutein (Pre) as shown in Table 3.

TABLE 3

| | Conventional Lutein (ng/mL) | Lutein SLP (ng/mL) |
|---|---|---|
| Pre | 59 ± 6 | 52 ± 7 |
| Post | 111 ± 14 | 345 ± 49 |
| Change | 52 ± 13 | 293 ± 50 |

p = 0.001 by unpaired t-test

PROBLEM: The state of the art on lutein bioavailability is not clear as to whether free or esterified lutein is better absorbed—both appear to be equally absorbed.

SOLUTION: Embodiments of this invention clarify the art and uses lutein ester (form found in nature) as the active component in order to achieve stable levels in the bloodstream as shown in Table 3, above.

PROBLEM: Lutein esters are typically found in nature as diesterified forms, with two fatty acid groups occupying the sites of the hydroxyl groups normally found in lutein, e.g., as lutein dipalmitate. However, most free lutein on the market requires a very high concentration which is not cost-effective, and is extracted with harsh/toxic solvents.

SOLUTION: Embodiments of the present invention allow for the use of the natural form (ester) without chemical reactions to de-esterify or use of toxic solvents.

Additional problems or issues encountered when formulating oral dosage forms of lipophilic compounds having at least one hydroxyl group addressed by the disclosed SLP oral delivery system include: the use of components not suitable for food use to address bioavailability and/or stability issues and the use of delivery systems that are not stable in gut.

PROBLEM: Solid Lipid Nanoparticles (SLN), liposomes etc. targeting lymphatic transport are often unstable in the gut. Improvements in oral bioavailability of nanoparticles as a result are often limited.

SOLUTION: Solid lipid particles (SLPs) that are an agglomeration of microparticles are stabilized in the varying pH and aqueous environment of the gut, allowing for lymphatic transport. The composition is a powder made of an agglomeration of SLP microparticles, with powder size being between about 20 and about 90 mesh, and with particle size in the agglomeration being in the range of about 5 to about 20 microns. The agglomeration of particles allows for less surface area exposed to the stomach acid and bile.

PROBLEM: Most products on the market either have failed in clinical efficacy trials, do not reach therapeutic blood levels, or require doses higher than practical in order to be effective.

SOLUTION: Clinical data on low doses of curcumin and boswellic acid SLPs is available demonstrating efficacy.

The oral delivery system and therapeutic compositions of embodiments of the present invention may be formulated into any suitable oral nutraceutical or pharmaceutical dosage forms including, but not limited to, tablets, capsules, powders, liquids, chews, gummies, etc. using standard excipients and formulation techniques in the industry.

### Example 3: Plasma Lutein Concentrations in Adult Subjects

This example demonstrates the increased bioavailabilty of lutein when paired with granules (SLCPs) of the present technology when compared to conventional lutein.

### Method

Study protocol was approved by the Ohio Health Institutional Review Board. Subjects were 12 males and females (six of each gender) aged 52 to 69, mean±SD of 57±3 for the standard lutein, and 59±6 for the novel lutein complex. Based on answers to an eligibility questionnaire, the accepted subjects were nonsmokers who were free from problems that cause widespread oxidant stress or cause problems with absorption of lipid nutrients. Also, based on answers to the questionnaire, subjects did not consume eggs, spinach, or kale more than four times a month, nor take lutein supplements.

Subjects were randomly assigned to either lutein ester or a solid-lipid particle (SLP) complexed lutein. The latter was supplied by Verdure Sciences, Noblesville, Ind., USA. The subjects took a single capsule of 10 mg lutein for 10 days (same mg of lutein/day/treatment, though different weights of total powder). Subjects were blinded to group assignment. The capsules were taken with a self-selected meal containing at least 200 calories of fat. The subjects provided a blood sample in a heparin containing tube before and after the 10-day supplementation period as well as 7 days after discontinuing the supplement. Plasma was separated by centrifugation for 30 min at 3000 rpm. Plasma lutein was determined by HPLC.

Changes within each supplement group were analyzed by paired t-test using http://www.fon.hum.uva.nl/Service/Statistics/Student_t_Test.html Group comparisons were done by unpaired t-test using http://www.fon.hum.uva.nl/Service/Statistics/2Sample_Student_t_Test.html

### Results

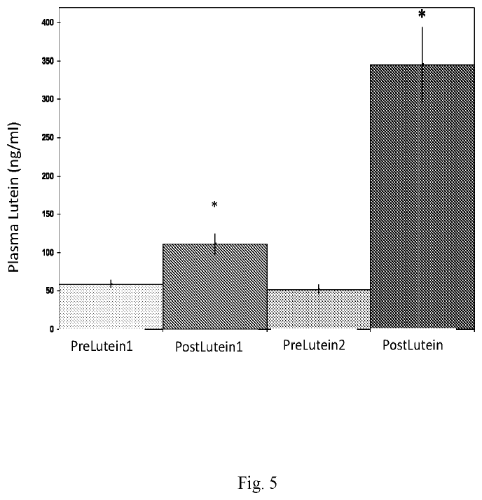

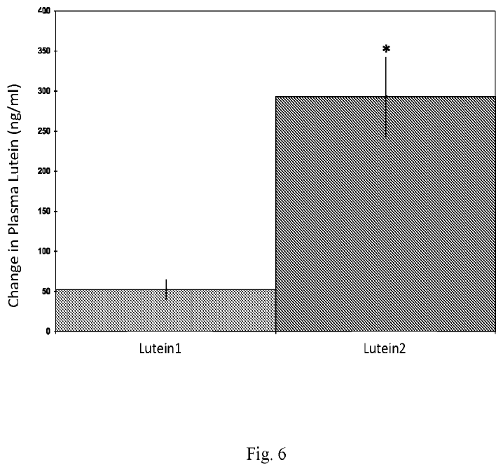

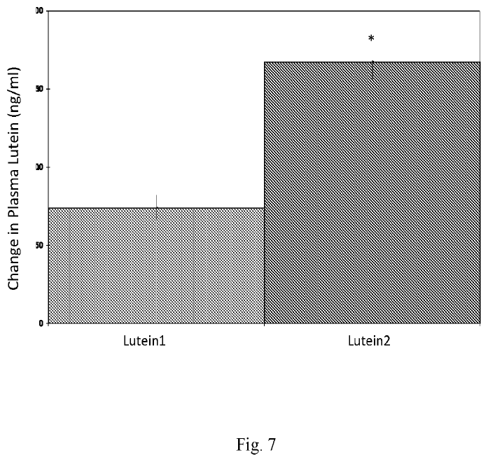

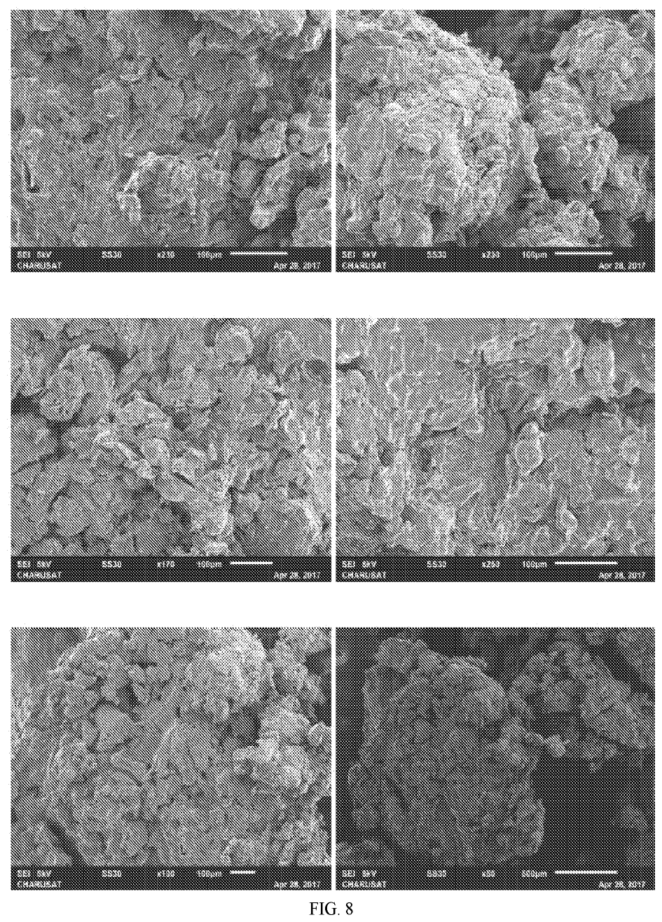

After 10 days of supplementation, both supplements produced highly significant increases in plasma lutein values (FIG. 5, p<0.001 for each treatment, paired t-test). The solid-lipid particle complexed lutein gave a significantly higher mean plasma lutein value than conventional lutein (p<0.001, unpaired t-test). The mean percent change versus pre-supplement values was 563% for the solid-lipid particle complexed lutein and 88% for the conventional lutein ester. If the data is expressed as the change in lutein concentrations, a significantly higher mean change was seen with the solid-lipid particle complexed lutein (FIG. 6, p<0.001, unpaired t-test). For both the conventional and new SLP lutein supplement, mean plasma lutein levels remained above baseline 7 days after supplementation (FIG. 5, pre-values vs FIG. 7, p<0.001, paired t-test). However, the solid-lipid particle complexed lutein gave a significantly higher mean plasma lutein value (FIG. 7, p<0.001, paired t-test). Thus, by three types of methodologies, plasma lutein concentrations responded to a significantly greater degree to the solid-lipid particle complexed lutein than to a conventional form of lutein.

In summary, a 10 day supplementation of solid-lipid particle complexed lutein produced far greater and statistically significant plasma levels as compared to lutein ester. Not to be bound by any particular theory, the higher plasma lutein concentrations produced by the solid-lipid particle complexed lutein is most likely due to better absorption from the GI tract.

### Example 4: Curcumin can Traverse the Blood-Retinal Barrier

It has been shown that curcumin when orally administered in formulations comprising SLPs can traverse the blood-ocular barrier. This was shown in a study done by the McCuster Alzheimer's Research Foundation, in which volunteers took curcumin supplement (curcumin SLPs, LONGVIDA® Optimized Curcumin, from Verdure Sciences). Retinal image fluorescence photography demonstrated that curcumin bound to and fluoresced the amyloid-beta plaques in volunteer's retinas, demonstrating that the curcumin SLP is able to traverse the blood-retinal barrier to bind to amyloid within the retina. (data not shown)

### Example 5: Retinal Inflammation in Aged Mice and the GFAP-IL6 Mice, a Model of Chronic Neuroinflammation

In the first part of this Example, GFAP-IL-6 transgenic mouse model is a viable model for studying chronic neuroinflammation with resulting neurodegeneration. The role of neuroinflammation in neurological disorders has stimulated the search for specific radiotracers targeting the peripheral benzodiazepine receptor (PBR), also known as the 18 kDA translocator protein (TSPO). TSPO is a marker of “active” brain inflammation using in vivo imaging such as Positron Emission Tomography (PET). TSPO may be a valuable diagnostic to detect activated microglia in the brain in vivo and monitor the progress of anti-inflammatory treatment.

Activated microglia can be directly associated with retinal inflammation. As such, activation of microglia can be used to directly observe retinal inflammation.

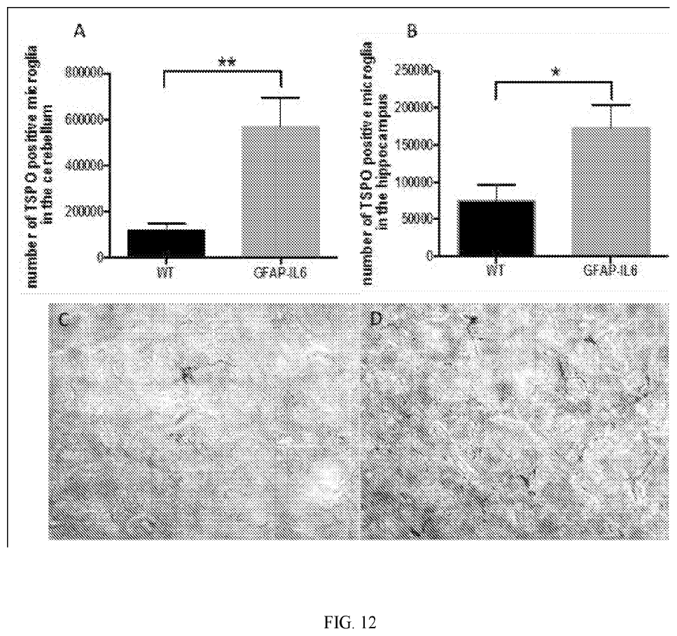

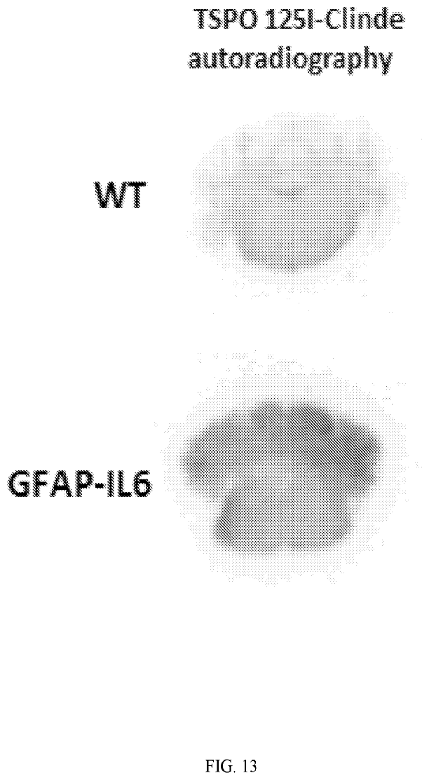

Experiments were performed to compare the number of TSPO microglia between WT and IL6 mice. Preliminary immunohistochemically results suggest a significant increase in TSPO activation in GFAP-IL6 mice, with a 4.75 times increase in the cerebellum of GFAP-IL6 mice and a 2.28 times increase in the hippocampus of GFAP-IL6 mice at 24 months of age (See FIG. 12). Autoradiography was also performed with the TSPO ligand 125I-Clinde in GFAP-IL6 mice compared to the WT control mice (See FIG. 13).

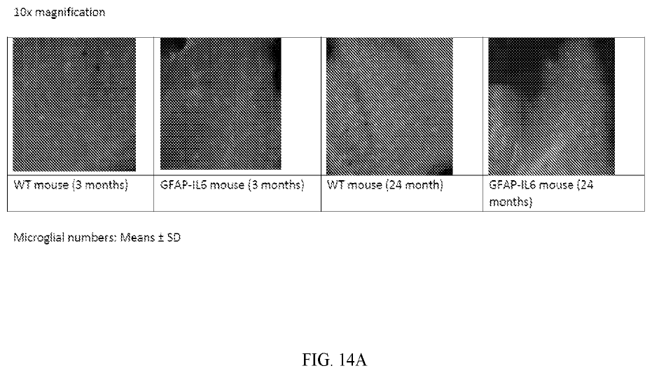

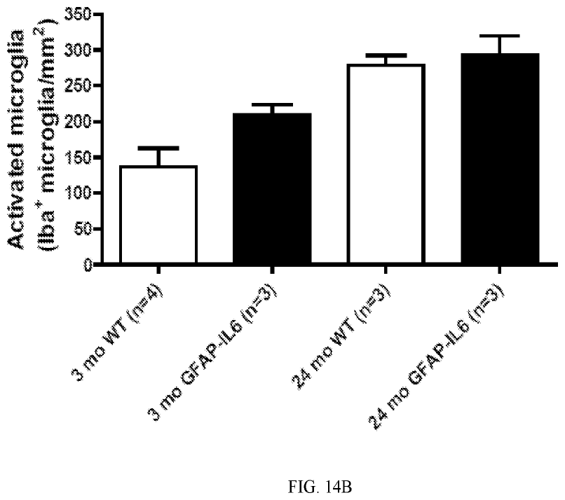

Further experiments show that retinal inflammation increases with age of mice. Four different groups of mice were analyzed: aged mice WT and GFAP-IL6 mice at 24 months and young mice WT and GFAP-IL6 at 3 months. Retinas (n=3-4) per group were analyzed, and the number of lba-1 positive microglia (marker of activated microglia) was determined. Results are shown in FIGS. 14A and 14B. In GRAP-IL6 mice, retinal inflammation is accelerated and can already be seen at 3 months of age.

An additional study will investigate two separate cohorts of 6 months to assess the performance of GFAP-IL6 mice compared in several behavior test to that of sex and age-matched non-transgenic WT controls (n=20 per group, genotype). Behavioral tests include tests for assessing fine motor coordination and reference memory. This study also includes immunohistological evaluation of microglial and astroglial activation to address neuroinflammation and neurodegeneration.

### Example 6: Fourier Transform Infrared Spectroscopy of Samples

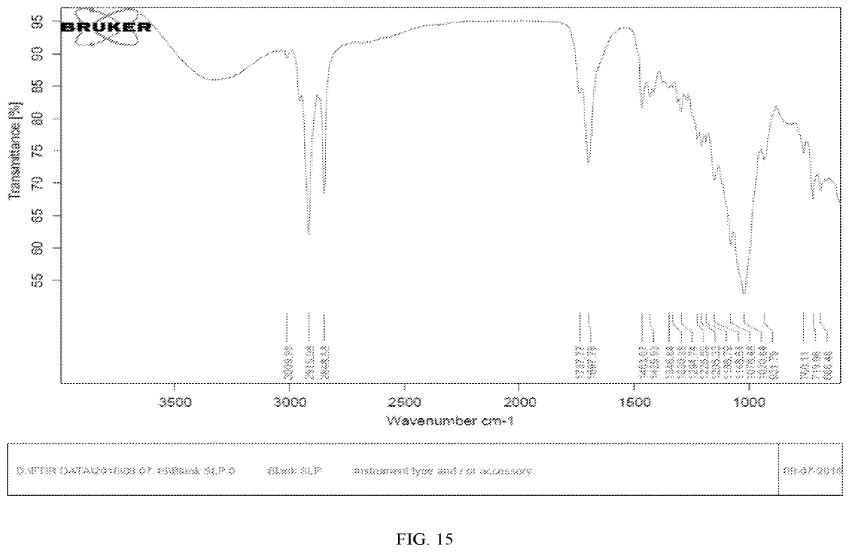

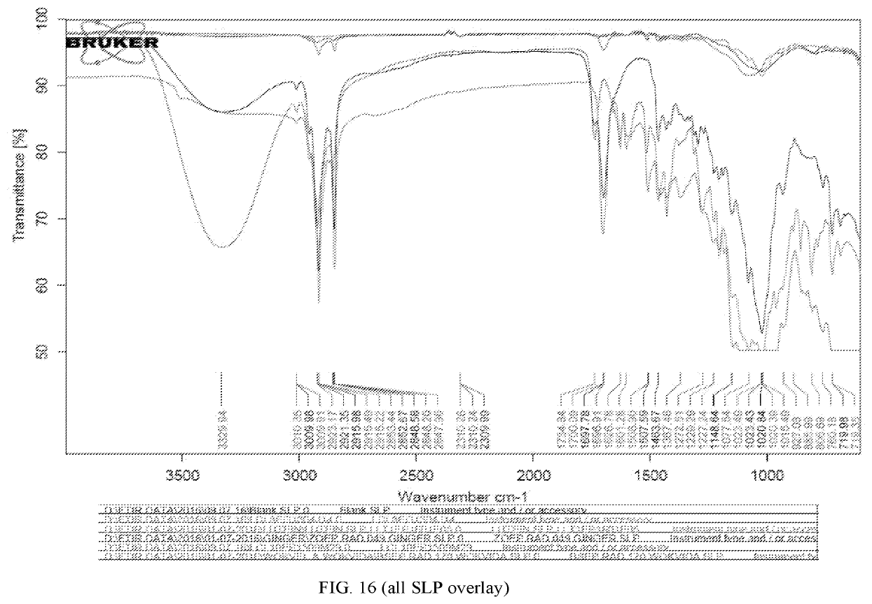

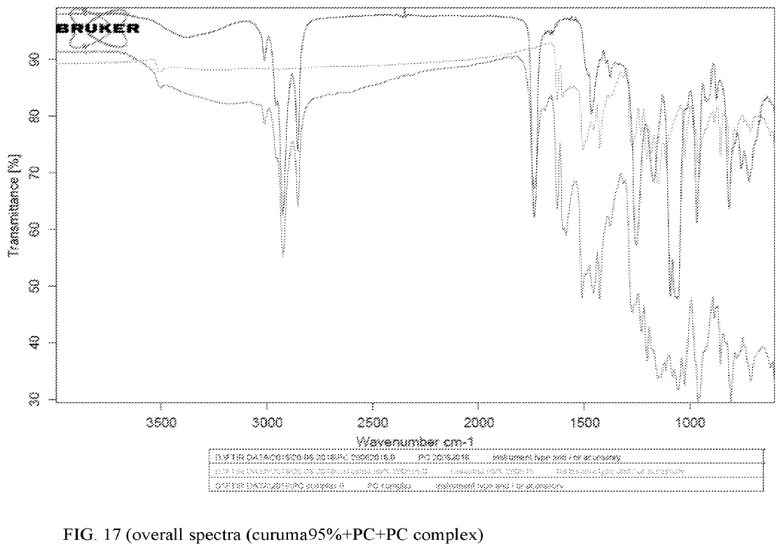

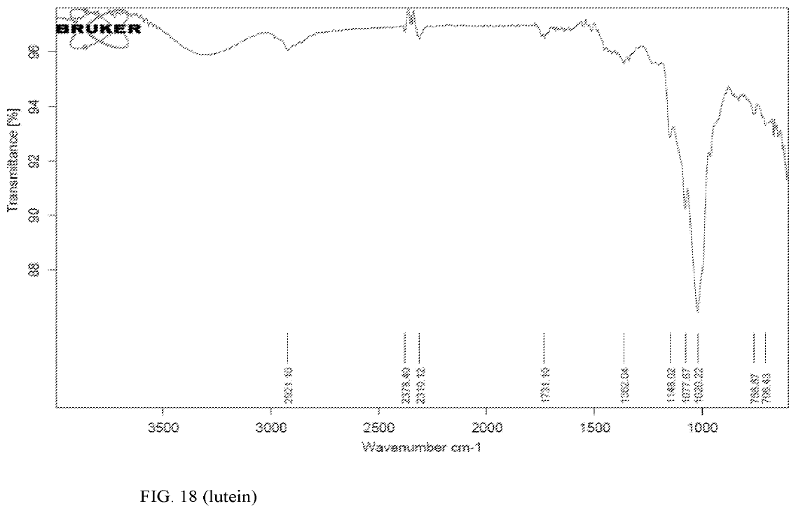

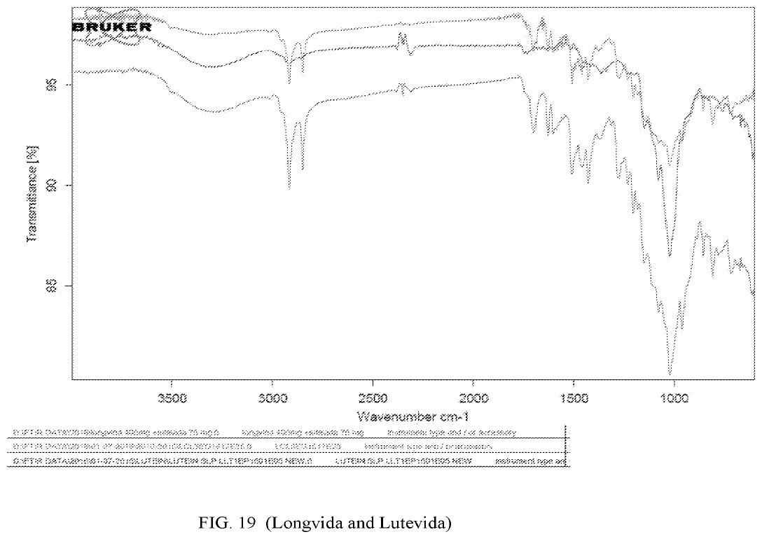

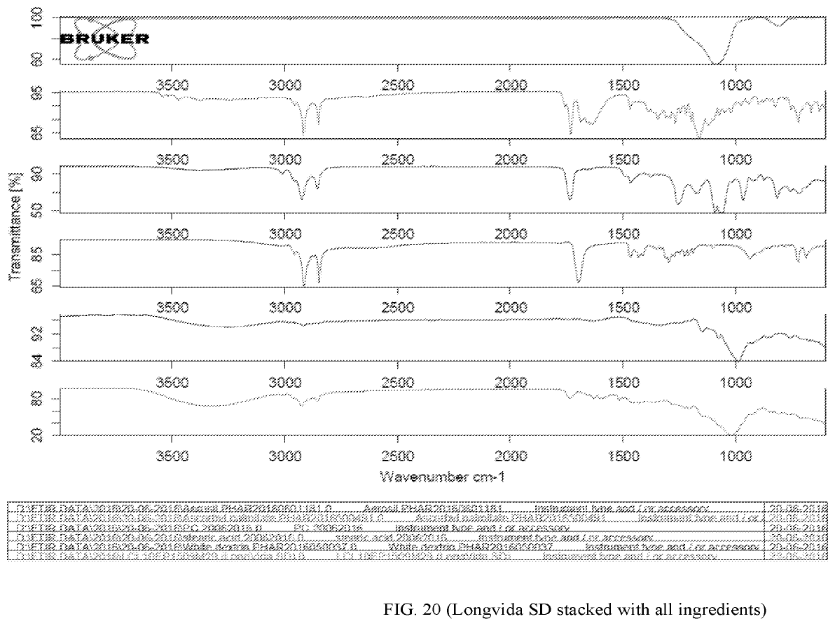

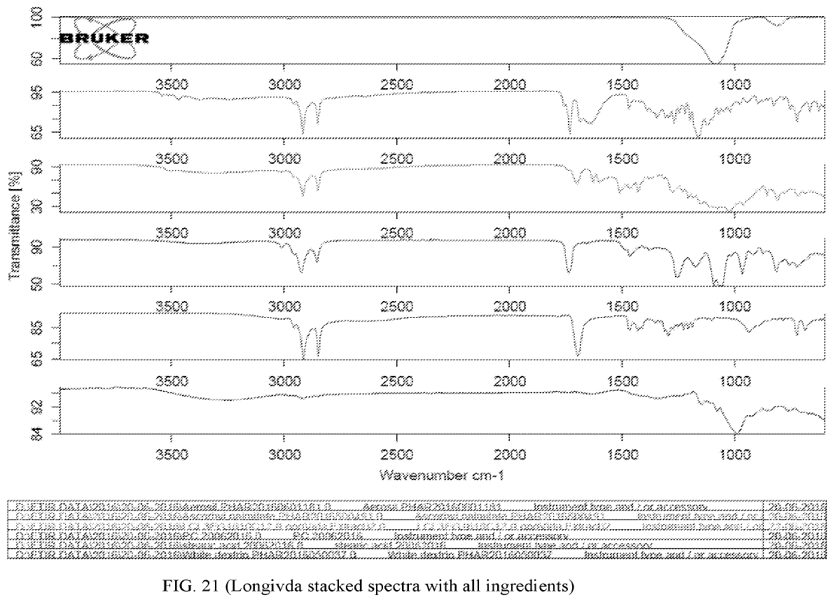

Fourier Transform-Infrared Spectroscopy (FTIR) was used to analyze the different curcumin and lutein SLP samples according to embodiments of the present invention. FTIR is an analytical technique used to identify organic and inorganic materials within a formulation. FTIR measures the absorption of infrared radiation by the sample material versus wavelength. The infrared absorption bands identify molecular components and structures. The FTIR of different formulations can be seen in FIGS. 15-21. [Any specific results or differences between the different formulations that can be described here?]

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

1. A method of treating, reducing or inhibiting retinal inflammation in a subject, the method comprising:
administering an effective amount of an enhanced oral bioavailable formulation of curcumin, lutein or a combination thereof, the enhanced oral bioavailable formulation comprising:
(a) curcumin, lutein or a combination thereof complexed with a solid lipid particle;
(b) a carrier granule comprising an agglomeration of the solid lipid particle embedded in, adhered to or both embedded in and adhered to, the carrier granule, wherein the solid lipid particle carrier granule complex is formulated for oral dosing.

2. A method of treating a retinal disease in a subject, the method comprising:
administering an effective amount of an enhanced oral bioavailable formulation of curcumin, lutein or a combination thereof, the enhanced oral bioavailable formulation comprising:
(a) curcumin, lutein or a combination thereof complexed with a solid lipid particle;
(b) a carrier granule comprising an agglomeration of the solid lipid particle embedded in, adhered to or both embedded in and adhered to, the carrier granule, wherein the solid lipid particle carrier granule complex is formulated for oral dosing.

3. The method of claim 2, wherein the retinal disease is selected from the group consisting of glaucoma, diabetic retinopathy, and retinal vein occlusion.

4. The method of claim 2, wherein the retinal disease is associated with retinal inflammation.

5. The method of claim 1, wherein the carrier granule has a particle size from about 150 to about 840 microns.

6. The method of claim 1, wherein the solid lipid particle has a particle size from about 5 to about 20 microns.

7. The method of claim 1, wherein the solid lipid particle comprises one or more long-chain lipid.

8. The method of claim 1, wherein the solid lipid particle comprises one or more long chain lipid selected from the group consisting of soy lecithin, phosphatidylcholine, stearic acid, and ascorbyl palmitate.

9. The method of claim 1, wherein the solid lipid particle comprises dextrin, silicone dioxide, or both.

10. The method of claim 1, wherein the ratio steric acid:phosphatidylcholine is in a range of about 1.25:1 to about 3.5:1.

11. The method of claim 1, wherein the solid lipid particle comprises a core comprising the active ingredient, phosphatidylcholine and ascorbyl palmitate, and a coating of stearic acid.

12. The method of claim 1, wherein the solid lipid particle is embedded in the carrier granule.

13. The method of claim 1, wherein the solid lipid particle is adhered to the surface of the carrier granule.

14. The method of claim 1, wherein the solid lipid particle is encased within the carrier granule.

15. The method of claim 1, wherein the carrier granule is formed in all or in part from fractured solid lipid microparticles.

16. The method of claim 1, wherein at least one of the carrier granule, solid lipid particle, and solid lipid particle carrier granule complex pass through the blood-ocular barrier.

17. The method of claim 1, wherein the formulation comprises:
about 15 to about 40% of curcumin, lutein, or a combination thereof;
about 7 to about 25% soya lecithin;
about 7 to about 30% maltodextrin;
about 1 to about 3% ascorbyl palmitate; and
about 0.3 to about 2% silicone dioxide.

18. The method of claim 17, wherein the soya lecithin is phosphatidylcholine.

19. The method of claim 1, wherein the formulation comprises:
about 10 to about 30% of curcumin, lutein, or a combination thereof;
about 10 to about 20% of phosphatidylcholine;
about 25 to about 35% stearic acid;
about 25 to about 40% dextrin;
about 1 to about 4% ascorbyl palmitate; and
about 0.1 to about 3% silicon dioxide.
